(19) United States
(12) Patent Application Publication
(10) Pub. No.: US 2013/0041163 A1
(43) Pub. Date: Feb. 14, 2013

(54) PRODUCTION OF ATORVASTATIN LOW IN LACTONE IMPURITIES

(71) Applicant: Ben De Lange, Munstergeleen (NL)

(72) Inventor: Ben De Lange, Munstergeleen (NL)

(73) Assignee: DSM Sinochem Pharmaceuticals Netherlands B.V., Delft (NL)

(21) Appl. No.: 13/642,258

(22) PCT Filed: Apr. 18, 2011
(86) PCT No.: PCT/EP2011/056094

§ 371 Date: Oct. 19, 2012

(30) Foreign Application Priority Data
Apr. 19, 2010 (EP) 10160281.1
Sep. 6, 2010 (EP) 10175441.4

(51) Int. Cl. C07D 207/34

(52) U.S. Cl. 548/537

(57) ABSTRACT

The present invention relates to a method for the production of atorvastatin having decreased levels of impurity (3) by means of a pH-controlling step.

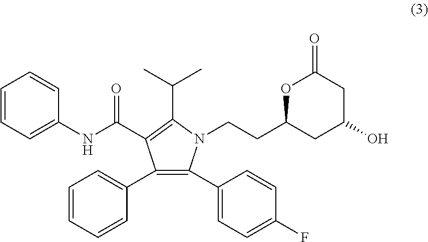

### FIELD OF THE INVENTION

The present invention relates to a method for the production of atorvastatin.

### BACKGROUND OF THE INVENTION

Atorvastatin ([R*,R*)]-2-(4-fluorophenyl)-8,6-dihydroxy-5-(1-methylethyl)-3-phenyl-4-[(phenylamino)carbonyl]-1H-pyrrole-1-heptanoic acid hemi calcium salt, (2)) is a pharmaceutical ingredient useful as an inhibitor of the enzyme 3-hydroxy-3-methylglutaryl-coenzyme A reductase (HMG-CoA reductase) and thus useful as a hypolipidemic and hypocholesterolemic agent.

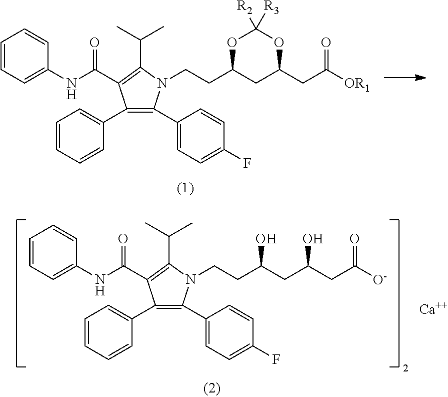

The preparation of atorvastatin is well known and the most common approach is based upon protection/deprotection of the carboxylic acid function as a tert-butyl ester ((1); R₁=—C(CH₃)₃) and of the diol functionality as an acetonide ((1); R₂=R₃=—CH₃). This has been described in WO 2008/129562 and in the many references cited therein.

As with any active pharmaceutical intermediate, also for atorvastatin strict control of impurities in the end product is of pivotal importance. The general reaction sequence described above utilizing tert-butyl ester protection of the carboxylic acid function has been investigated intensively. The unique chemical nature of the tert-butyl ester function has leads to a unique spectrum of impurities, both in terms of chemical structure as well as in terms of relative amounts. Various solutions have been given for reducing the amounts of impurities in the documents mentioned above. However, all these solutions apply to tert-butyl ester protection strategies.

Recently, another type of protection has been disclosed in WO 2009/019561, namely the use of the iso-propyl group for carboxylic acid protection ((1); R₁=—CH(CH₃)₂). The differences in chemical reactivity of this group as compared to the tert-butyl ester function under the prior art deprotection conditions leads to differences in impurity profiles calling for different solutions.

### DETAILED DESCRIPTION OF THE INVENTION

In a first aspect of the invention, the production of atorvastatin hemi calcium salt of formula (2) from a compound of general formula (1) wherein R₁=—CH(CH₃)₂ and R₂ and R₃ are independently chosen from the list consisting of ethyl, methyl and propyl or wherein R₂ and R₃ form a cyclopentylidene or cyclohexylidene ring, is achieved by the steps of:

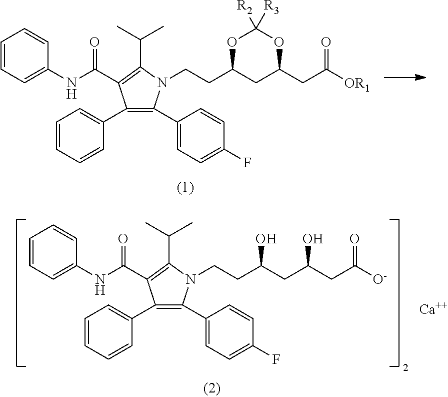

(a) Treating a solution of said compound of general formula (1) in a first solvent with an acid;
(b) Treating the mixture obtained in step (a) with an alkali metal hydroxide;
(c) Treating the washed mixture obtained in step (b) with a calcium salt or with calcium hydroxide,

Suitable first solvents include acetonitrile, alcohols, cyclic ethers such as dioxane and tetrahydrofurane and ketones such as acetone and mixtures thereof. The first solvent may be an alcohol such as ethanol, iso-propanol, methanol and propanol. The acid used in step (a) may be an inorganic acid such as hydrobromic acid, hydrochloric acid, nitric acid, phosphoric acid or sulfuric acid. The amount of alkali metal hydroxide added in step (b) is such that the pH of the mixture is raised to a value between 11 and 13, preferably between 11.5 and 12.5. Suitable alkali metal hydroxides are lithium hydroxide, potassium hydroxide and sodium hydroxide. The calcium salt used in step (c) may be calcium acetate. Seed crystals of atorvastatin hemi calcium salt crystal form I may be added prior to the addition of the calcium salt in order to facilitate crystallization.

Atorvastatin hemi calcium salt precipitates after step (d) and can be isolated by filtration, centrifugation or other techniques known to the skilled person. Optionally the product thus obtained is further purified by re-slurrying in aqueous environment, preferably in water followed by isolation and drying of the crystals.

In the course of the process described above impurity (3) is formed.

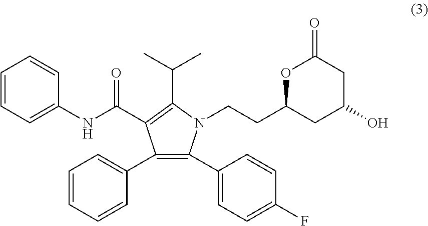

Unfortunately the amounts wherein this impurity is present in the final product may be as high as 0.1% which is unwanted in view of USP and/or European Pharmacopoeia (Ph. Eur.) requirements. Surprisingly it was found that the amount of impurity (3) could be lowered dramatically by lowering the pH of the mixture obtained after step (b) to a value not lower than 7.5, or not lower than 7.9, or not lower than 8.0. This can be achieved by adding an acid to the mixture obtained in step (b) or by adding an acid to the mixture obtained after step (c). In principle the commonly used organic and inorganic acids are suitable for the purpose of the present invention although in our hands acetic acid gave still more superior results. By applying the above measure atorvastatin hemi calcium salt crystal form I is obtained with levels of impurity (3) as low as from 0.0001% to 0.06%.

It has been found that in specific cases it may be advantageous to perform a washing step with a second solvent in between process steps (b) and (c). Said second solvent may be an ether such as, for example, dibutyl ether, diethyl ether, methyl ether, methyl tert-butylether or an ester such as ethyl acetate or iso-propyl acetate or hydrocarbons such as toluene or petroleum ether.

In a second aspect of the invention there is disclosed is a composition comprising atorvastatin hemi calcium salt and from 0.0001% to 0.06% by weight of a compound of general formula (3).

### EXAMPLES

### Example 1

### Preparation of Atorvastatin Calcium from 2-((4R,6R)-6-(2-(3-(phenylcarbamoyl)-5-(4-fluorophenyl)-2-isopropyl-4-phenyl-1H-pyrrol-1-yl)ethyl)-2,2-di methyl-1,3-dioxan-4-yl)acetic Acid Isopropyl Ester ((1); R₁=—CH(CH₃)₂, R₂ and R₃=CH₃)

2-((4R,6R)-6-(2-(3-(phenylcarbamoyl)-5-(4-fluorophenyl)-2-isopropyl-4-phenyl-1H-pyrrol-1-ypethyl)-2,2-dimethyl-1,3-dioxan-4-yl)acetic acid isopropyl ester (75 kg, 117 mol) was added to 1125 L of methanol and stirred at 35-37° C. until clarity. After cooling to 25-27° C. aqueous HCl (made from 19 kg concentrated HCl and 64 L of water) was added and the reaction was stirred for 2 h at 25-27° C. The mixture was concentrated under vacuum in 3.5 h at 20-22° C. to about 50% of its original volume. Then 825 L of methanol was added and the mixture stirred for 45 min. HPLC analysis revealed the starting material to be less than 0.03%. Aqueous NaOH (made from 15 kg NaOH and 560 L of water) was added keeping the temperature below 30° C. to give a pH of 12.2 and stirring was continued for 2 h. The reaction mixture was concentrated to about 900 L under vacuum at a temperature of 20-22° C. in 5 h. Next, 750 L of water and 450 L of methyl-t-butyl ether were added and stirred for 15 minutes after which phases were separated. Methyl-t-butyl ether (450 L) was added and stirred for 15 min. after which phases were separated. After heating the aqueous layer to 35-37° C., 7.5 kg active carbon was added followed by stirring for 30 min. The reaction mixture was filtered through a Hyflo bed and the carbon/hyflo bed washed with water/methanol (135 L water/15 L methanol). The resulting solution was concentrated under vacuum, followed by addition of 150 L of water and 37.5 L of methyl-t-butyl ether. The temperature was increased to 45-47° C. and the pH adjusted to 8.6-8.8 with aqueous acetic acid (3 L acetic acid in 60 L of water). After heating the reaction mixture until 47-50° C., 7.5 kg of atorvastatin calcium polymorph I seed was added, followed by addition in 1 h of a solution of 14.5 kg Ca-acetate in 375 L of water. The mixture was heated to 55-58° C. and maintained at this temperature for 30 min. The slurry was then cooled to 40-45° C. and stirred for 3 h. The solid was isolated by centrifugation and the obtained wet-cake re-slurried in 1125 L of water at 40-45° C. After stirring for 1 h, the solid was isolated by centrifugation and dried under vacuum at 50-55° C. Weight 63.5 kg. If required, the material can be milled, blended and/or micronized. Impurity: Lactone (3): 0.05%.

### Comparative Example 2 (pH at 7.2)

### Preparation of Atorvastatin Calcium from 2-((4R,6R)-6-(2-(3-(phenylcarbamoyl)-5-(4-fluorophenyl)-2-isopropyl-4-phenyl-1H-pyrrol-1-yl)ethyl)-2,2-dimethyl-1,3-dioxan-4-yl)acetic Acid Isopropyl Ester ((1); R₁=—CH(CH₃)₂, R₂ and R₃=—CH₃)

2-((4R,6R)-6-(2-(3-(phenylcarbamoyl)-5-(4-fluorophenyl)-2-isopropyl-4-phenyl-1H-pyrrol-1-ypethyl)-2,2-dimethyl-1,3-dioxan-4-yl)acetic acid isopropyl ester (90 kg, 141 mol) was added to 1350 L of methanol and stirred at 35-37° C. until clarity followed by cooling to 25-27° C. Then aqueous HCl (made from 22.8 kg concentrated HCl and 77 L of water) was added. The reaction was stirred for 2 h at 25-27° C. The mixture was then concentrated under vacuum in about 16 h at 29-30° C. to about 30% of its original volume. Then 825 L of methanol was added and the mixture was concentrated under vacuum in 5 h at 29-30° C. to ˜40% of its original volume. Methanol (900 L) was charged followed by 620 L of aqueous 0.6 N NaOH keeping the temperature below 30° C. to give a pH of not less than 12 (actual temperature 26° C. and pH 12.4) and stirring was continued for 2 h. The reaction mixture was concentrated (˜900 L was distilled) under vacuum in 8 h at 29-30° C. Next, 810 L of water and 540 L of methyl-t-butyl ether were added and stirred for 15 minutes after which phases were separated. The aqueous layer was heated to 35-37° C., 5.0 kg active carbon added and stirred for 30 min. The reaction mixture was filtered through a Hyflo bed and the carbon/Hyflo bed washed with water/methanol (80 L water/10 L methanol). To the solution, 22 L of ethylacetate was added and stirred for 1 h. The pH was initially 8.3 but went down to approximately 7.2 during the next process steps. After heating the reaction mixture until 47-50° C., 9.0 kg of atorvastatin calcium polymorph I seed was added, followed by addition in 1 h of a solution of 14.0 kg Ca-acetate in 270 L of water. The mixture was heated to 55-58° C. and maintained at this temperature for 30 minutes. The slurry was then cooled to 40-45° C. and stirred for 3 h. The solid was isolated by centrifugation and the wet-cake re-slurried in 900 L of water at 40-45° C. After stirring for 1 h, the solid was isolated by centrifugation and dried under vacuum at 50-55° C. Weight 75.9 kg. If required, the material can be milled, blended and/or micronized. Impurity: Lactone (3): 0.17%

### Comparative Example 3 (pH 7.1 During Extractions Steps)

### Preparation of Atorvastatin Calcium from 2-((4R,6R)-6-(2-(3-(phenylcarbamoyI)-5-(4-fluorophenyl)-2-isopropyl-4-phenyl-1H-pyrrol-1-yl)ethyl)-2,2-dimethyl-1,3-dioxan-4-yl)acetic Acid Isopropyl Ester ((1); R₁=—CH(CH₃)₂, R₂ and R₃=—CH₃)

2-((4R,6R)-6-(2-(3-(phenylcarbamoyl)-5-(4-fluorophenyl)-2-isopropyl-4-phenyl-1H-pyrrol-1-ypethyl)-2,2-dimethyl-1,3-dioxan-4-yl)acetic acid isopropyl ester (30 g, 31 mmol) was added to 450 mL of methanol and at 33-35° C. until a clear solution was obtained followed by cooling to 26-28° C. Then 36 mL of 2.2 N aqueous HCl was added in 15 min and the resulting reaction mixture stirred for 2 h at 26-28° C., when HPLC analysis revealed the starting material to be less than 0.1%. To the mixture, 205 mL of 0.6 N aqueous NaOH was added in 1 h keeping the temperature below 30° C. The pH was 12.3. After stirring for 2 h, the clear solution was concentrated under vacuum at 27-29° C. until a slurry was obtained. Then 300 mL of water and 180 mL of methyl-t-butyl ether were added. The phases were separated. The aqueous layer was extracted with a mixture of ethylacetate/cyclohexane (240 mL ethylacetate and 240 mL cyclohexane, 50/50). The phases were separated. The pH of the aqueous phase was ˜7.1. Thereafter, the aqueous phase was treated with 3.0 g active carbon. After filtration of the carbon, the reaction mixture was heated until 45-50° C., 60 mL of H₂O was added and the pH adjusted to 8.5 with 0.6 N aqueous NaOH. Then, 3.0 g of Atorvastatin Calcium Polymorph I seed was added, followed by addition in 1 h of a solution of 5.5 g Ca-acetate in 150 mL of water. The mixture was heated to 55-58° C. and maintained at this temperature for 30 minutes. The slurry was cooled to 40-45° C. and stirred for 3 h. The solid was isolated by filtration and the wet-cake re-slurried in 400 mL of water. The slurry was heated to 40° C., stirred for 1 h and filtered. The white solid was dried at 50-55° C. Weight 22.8 g. Impurities: Lactone (3): 0.18%.

TABLE
Summary of results

| Example | pH control | Lactone (3) |
|---|---|---|
| 1 | 8.6-8.8 | 0.05 |
| 2 | 8.3 → 7.2 | 0.17 |
| 3 | 7.1 → 8.5 | 0.18 |

1. Method for the production of atorvastatin hemi calcium salt of formula (2) from a compound of general formula (1) wherein R₁=—CH(CH₃)₂ and R₂ and R₃ are independently chosen from the list consisting of ethyl, methyl and propyl or wherein R₂ and R₃ form a cyclopentylidene or cyclohexylidene ring, comprising the steps of: 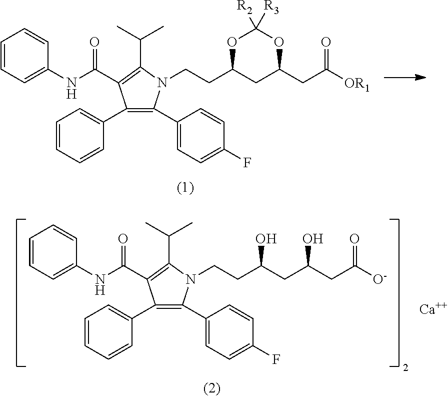
(a) Treating a solution of said compound of general formula (1) in a first solvent with a first acid;
(b) Treating the mixture obtained in step (a) with an alkali metal hydroxide;
(c) Treating the washed mixture obtained in step (b) with a calcium salt or with calcium hydroxide,
characterized in that a second acid is added after step (b) or after step (c) until the pH of the mixture is between 7.5 and 9.0.

2. Method according to claim 1 wherein said pH obtained after adding said second acid is between 8.0 and 8.5.

3. Method according to claim 1 wherein said second acid is acetic acid.

4. Method according to claim 1 wherein both R₂ and R₃ are methyl.

5. Method according to claim 1 wherein said first solvent is an alcohol.

6. Method according to claim 1 wherein said first solvent is methanol.

7. Method according to claim 1 wherein the mixture obtained after step (b) is washed with a second solvent prior to step (c).

8. Method according to claim 7 wherein said second solvent is an ether.

9. A composition comprising atorvastatin hemi calcium salt and from 0.0001% to 0.06% by weight of a compound of general formula (3) [chemical structure]
